(19) United States
(12) Patent Application Publication
(10) Pub. No.: US 2022/0040398 A1
(43) Pub. Date: Feb. 10, 2022

(54) DUAL CANNULATED SUCTION AND DELIVERY DEVICE

(71) Applicant: DAS Innovations Pty Ltd, Darling Point (AU)

(72) Inventors: Samuel Grasso, Bondi (AU); Duncan de Montfort, Bondi (AU); Arj Ananda, Homebush (AU)

(73) Assignee: DAS Innovations Pty Ltd, Darling Point (AU)

(21) Appl. No.: 17/508,246

(22) Filed: Oct. 22, 2021

Related U.S. Application Data
(63) Continuation of application No. PCT/AU2020/050396, filed on Apr. 24, 2020.

(30) Foreign Application Priority Data
Apr. 26, 2019 (AU) 2019901414

(51) Int. Cl. A61M 1/00; A61M 13/00

(52) U.S. Cl. CPC A61M 1/774; A61M 1/842; A61M 2202/064; A61M 1/86; A61M 13/00; A61M 1/85

(57) ABSTRACT

A medical device related to suction and delivery of components at a surgical site, the device comprising: a shaft body having a suction channel for suctioning a first component from a site, and a delivery channel associated with the shaft body for delivering a second component to the site.

### CROSS-REFERENCES TO RELATED APPLICATIONS

This application is a continuation of International Application No. PCT/AU2020/050396, filed Apr. 24, 2020, which claims priority to Australian Application No. 2019901414, filed Apr. 26, 2019. Each of the PCT/AU2020/050396 and AU 2019901414 applications are incorporated by reference herein in their entireties.

### BACKGROUND OF THE INVENTION

This disclosure relates to a medical surgical suction device.

A range of surgical procedures require the removal of fluids, debris, and tissue material from a surgical site of a patient's body. The removal is frequently carried out using suction devices.

Surgical suction devices are primarily used in medical procedures involving a removal of bodily fluid, debris, coagula, and any other element which obstruct the surgical site. The surgical suction device is used to keep the operating field clear of obstructing elements, and thus visible to the surgeon.

Surgical suction devices having means for variation of the suction are known, these are rigid one-piece instruments having straight-line elongated probe and suction members and requiring replacement of the entire instrument to change the size or length of the probe and suction members. Some surgical suction devices may include a suction tip coupled to suction tubing which is in turn coupled to a wall suction unit or the like. Some examples of existing suction tips include, an elongated single cannula for drawing the fluids or tissues from the surgical site into a suction handle which also provides for connection to the suction tubing. The cannula may be exchangeable to allow different shapes to be used depending on the particular surgery and application. The distal ends of these instruments tend to be sharp and capable of injury to delicate tissue. This may cause complexity in the surgical procedure.

In medical procedures such as cardiotomy operations usually, the blood is recycled through the suction device into a so called cardiotomy reservoir, which serves as a blood storage and filtering means, so that the blood may be re-infused to the patient during or after the operation. This reduces the amount of donated blood which has to be administered to the patient. However, in the heat of surgery, the surgeon's request for more suction usually causes the pump technician to merely turn the suction pump on and allow it to run constantly. As a result, haemolysis of the blood may occur with the creation of clots and other blood debris along with the formation of air microbubbles which are difficult to remove from the blood, and they are undesirable for re-infusion to the patient.

In addition, sometimes in minimally invasive surgery it is desired to deliver a sterile solution or some medications at an operative site and then to aspirate fluids from the surgical site under suction. The aspirated fluids can include, not only the sterile solution but also blood and other body fluids, tissue, debris, and fragments. Such a dual requirement of an operative environment poses significant design hurdles. In some surgeries suction of undesirable fluid and debris is required simultaneously.

There are several techniques used by health care professionals to use suction and delivery of components from and to the surgical sites by using two different devices. One device for the suction, and one for the delivery.

It is to be understood that, if any prior art is referred to herein, such reference does not constitute an admission that the prior art forms a part of the common general knowledge in the art, in Australia or any other country.

### BRIEF SUMMARY OF THE INVENTION

Disclosed is a medical device related to suction and delivery of components at a surgical site, the device comprising: a shaft body having a suction channel for suctioning a first component from a site, and a delivery channel associated with the shaft body for delivering a second component to the site. The device may be configured such that in use during surgery, the first component is removed from the site through the suction channel, and the second component is delivered to the surgical site through the delivery channel.

More particularly, the device may be in the form of a dual cannulated suction and delivery device applicable to medical instruments through which blood, coagula, debris and any other element the surgeon may find during a surgery procedure is obstructing the surgical site may be removed, and haemostatic solutions may be delivered to the surgical site simultaneously. The device can be used for various procedures including those performed in nasal and sinus cavities as well as in abdominal, thoracic, intra cranial, oral, and genitourinary system. It may also be used in dental applications. For surgeons, this device would be of great benefit allowing use of a single device for both suction and delivery of components. Use of the device may give flexibility to a surgeon to simultaneously deliver and remove the components during surgery. This has benefits for efficiency and ease of use. and may substantially reduce post-surgical complications.

In some forms the device allows for access to difficult anatomy due to the presence of a malleable component at the end of the shaft body.

### BRIEF DESCRIPTION OF THE DRAWINGS

Notwithstanding any other forms that may fall within the scope of the disclosure, specific embodiments will now be described, by way of example only, with reference to the accompanying drawings in which:

FIG. 1 shows a top view of an embodiment of the fluid transfer device;

FIG. 2 shows a side view of the device of FIG. 1;

FIG. 3 shows a bottom of the device of FIG. 1;

FIG. 4 shows an end view of the device of FIG. 1;

FIG. 5 shows a top view of a shaft of one embodiment of FIG. 1;

FIG. 6 shows a side perspective view of FIG. 5;

FIG. 7 shows a cross-section view of one embodiment AA of FIG. 5;

FIG. 8 shows a cross section view of FIG. 5;

FIG. 9 shows a top view of one embodiment of FIG. 1;

FIG. 10 shows a cross-section view of FIG. 9;

FIG. 11 shows a top view of a second embodiment of the fluid transfer device;

FIG. 12 shows a side view of the device of FIG. 11;

FIG. 13 shows a bottom view of the device of FIG. 11;

FIG. 14 shows an end view of the device of FIG. 11;

FIG. 15 shows a top view of a shaft of a second embodiment of the disclosure;

FIG. 16 shows a cross- section view of one embodiment AA of FIG. 15;

FIG. 17 shows a longitudinal cross-section of FIG. 15;

FIG. 18 shows a side perspective view of FIG. 15.

### DETAILED DESCRIPTION OF THE INVENTION

In the following detailed description, reference is made to accompanying drawings which form a part of the detailed description. The illustrative embodiments described in the detailed description, depicted in the drawings and defined in the claims, are not intended to be limiting. Other embodiments may be utilised, and other changes may be made without departing from the spirit or scope of the subject matter presented. It will be readily understood that the aspects of the present disclosure, as generally described herein and illustrated in the drawings can be arranged, substituted, combined, separated, and designed in a wide variety of different configurations, all of which are contemplated in this disclosure.

Disclosed broadly is a medical device related to suction and delivery of components at a surgical site, the device comprising: a shaft with a suction channel for suctioning a first component from a site, and a delivery channel associated with the shaft for delivering second component to the site. The device may be configured such that in use during surgery, the first component is removed from the site through the suction channel, and the second component is delivered to the surgical site through the delivery channel.

In some forms, the device comprises a multichambered shaft. In some forms, suction and delivery may occur simultaneously. In some forms the user has independent control of suction and delivery.

In some forms, the first component comprises any of a fluid, a solid, or a semi solid mixture. In some forms, the second component comprises a haemostatic solution in the form of a fluid or solid powder.

In some forms, the device comprises a suction connector configured to be engaged with the suction channel to effect suction through the suction channel.

In some forms, the device comprises a delivery connector at a delivery point to be engaged with the delivery channel to effect delivery through the delivery channel.

In some forms, the device comprises a delivery connector at a delivery point to be engaged with the delivery channel to effect delivery through the delivery channel.

In some forms, the suction channel and the delivery channel comprise hollow closed sections. In some forms, the closed sections are located within the shaft.

In some forms, the shaft includes fenestrations at the leading end to assist the suction and collection of blood from surrounding area.

In some forms, the shaft further comprises a flywheel for rotating the shaft with respect to the suction connector.

In some forms, the multichambered shaft comprises colour coded sections. The colour coded sections distinguish between the delivery chamber and the suction chamber.

In some forms the device comprises a malleable component at the end of the multichambered shaft to allow for flexibility and bending during use difficult anatomy.

In some forms, the device can be a single use device.

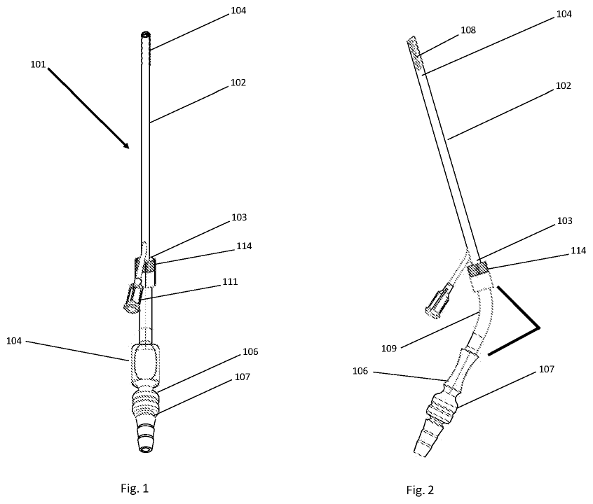

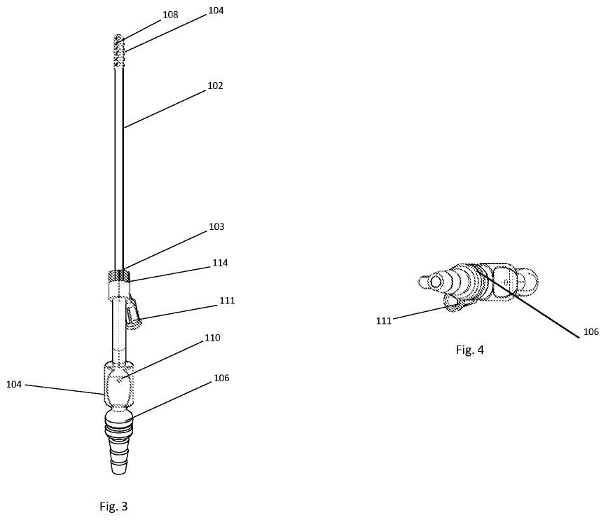

Referring now to FIGS. 1-4, disclosed is a medical device 101 for suction and delivery. The device 101 comprises an elongate shaft 102 extending from a connection end 103 to a leading end 104. The shaft in the illustrated form is generally tubular and extends in a substantially straight line between the connection end 103 and the leading end 104. However, it will be understood that variously shaped shafts may be utilised within the device.,

A suction connector 106 is engaged with the connection end 103 and is configured to attach the device with a source of suction (not illustrated). The suction connector 106 comprises a suction plug 107 having an intermediate suction attachment 109 which is configured to engage the shaft 102 with the suction connector 106. The suction plug 107 includes a suction hole 110 which is configured to allow a user to block the suction hole with a thumb.

In some forms, the shaft 102 and the suction connector 106 are angled with respect to another using the intermediate suction attachment 109. This is shown best in FIG. 2. The shaft 102 and the connector 106 are turned away from one another through between 30 and 60 degrees.

A delivery point 111 is located on the device to allow delivery to the shaft. The delivery point may include an injection point or similar attachment.

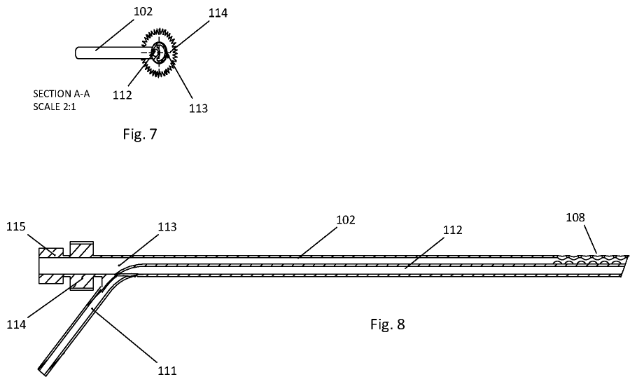

Referring now to FIGS. 5 through 8, in some forms, the shaft 102 is an elongated member with variation in lengths from 5 cm to 150 cm. In some forms, the length varies from 10 cm to 50 cm. In some forms the diameter of the shaft varies from 0.5 mm to 15 mm.

In the illustrated form, the shaft 102 comprises an elongate body extending between the connection end 103 and the leading end 104. A delivery point 111 is located intermediate the connection end 103 and the leading end 104 and in this form is located proximal the connection end 103. The delivery point in the illustrated form comprises and angled tube extending outwardly from the shaft 102.

The shaft further comprises a fly wheel 114. The fly wheel 114 allows for rotation of the shaft. This allows the shaft 102 to be rotated with respect to the suction connector 103. In some forms, shaft 102 comprises a shaft stopper and rotator mechanism.

In some forms, the shaft 102 can rotate up to 360 degrees in either clockwise or anti clockwise directions giving flexibility to surgeons to deliver and remove components from the surgical site simultaneously.

As shown best in the cross-section in FIG. 8, in some forms, the shaft 102 further comprises hollow closed section flow channels contained within the shaft 102. In the illustrated form a delivery flow channel 112 is configured for the delivery of components to the operational site. The delivery flow channel 112 is hollow and closed and has a circular or oval cross section but it will be clear that alternative configurations are available. The delivery flow channel 112 extends from the delivery point 111 to the leading end 104 and has a consistent cross section.

The remainder of the interior of the shaft 102 comprises a suction flow channel 113 for the suction of the components from the surgical site. In the illustrated form the suction flow channel comprises an arched hollow closed section located around the delivery flow channel. However it will be clear that the delivery and suction flow channels may each be round tubes, may each be interior the shaft or may be engaged to one another and extend parallel to one another. Alternative configurations that allow for a delivery flow channel and a suction flow channel to extend from a leading end in a single device also fall within the scope of the disclosure.

In the illustrated form the suction flow channel extends from the suction connector 106 to the leading end and for a substantial portion of the shaft 102, extends in line with the delivery flow channel. Both the flow channels may extend up to the full length of the shaft from the leading end.

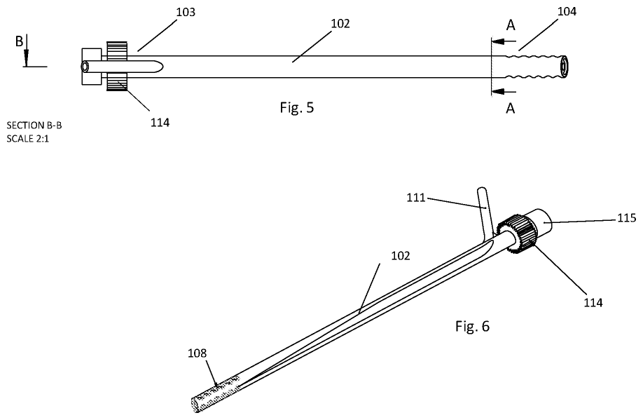

In the illustrated form, the shaft 102 comprises a plurality of fenestrations 108 at the leading end as seen best in FIGS. 2 and 6. The fenestrations are of round shape, but they could be of any other shape and/or sizes. The fenestrations assist the suction and resist blockage at one point of the leading end.

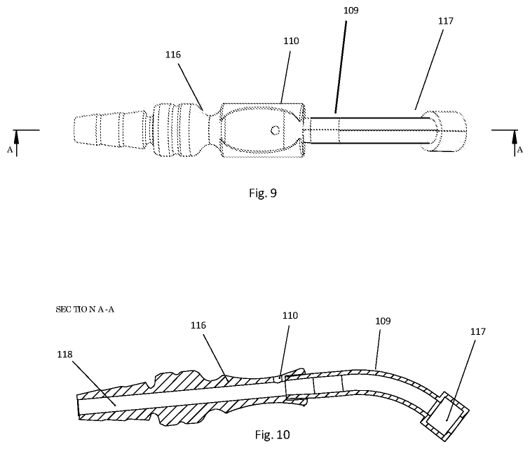

Referring to FIGS. 9 and 10, the suction connector 106 comprises a hollow suction chamber 118, a tool handle 116 as best seen in FIG. 10. The intermediate suction attachment 109 includes a hollow section and extends from a chamber 117 to a suction connector end.

The chamber 117 may allow for shaft rotation of the shaft with respect to the suction connector to allow for better angles and ease of use.

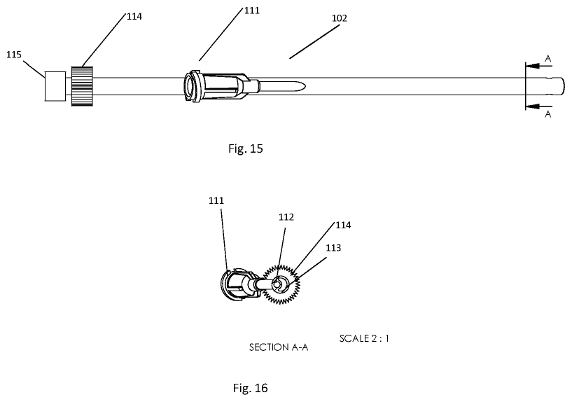

Referring to FIGS. 11-18, disclosed is a medical device 101 similar to that shown in FIGS. 1-4. The device 101 comprises an elongate shaft 102 extending from a connection end 103 to a leading end 104.

A suction connector 106 is engaged with the connection end 103 and is configured to attach the device with a source of suction (not illustrated). The suction connector 106 comprises a suction plug 107 having an intermediate suction attachment 109 which is configured to engage the shaft 102 with the suction connector 106. The suction plug 107 includes a suction hole 110 which is configured to allow a user to block the suction hole with a thumb.

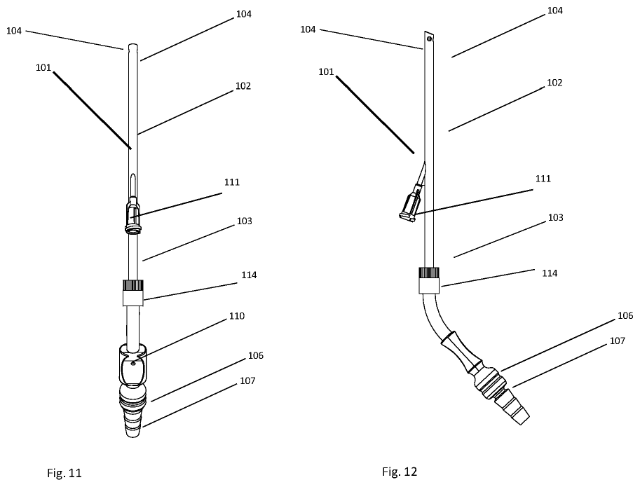

In some forms, the shaft 102 and the suction connector 106 are angled with respect to another using the intermediate suction attachment 109. This is shown best in FIG. 12. The shaft 102 and the connector 106 are turned away from one another through between 30 and 60 degrees.

A delivery point 111 is located on the device to allow delivery to the shaft. The delivery point may include an injection point or similar attachment. The delivery point 111 in this embodiment is located intermediate the leading end 104 and the connection end 103 of the shaft 102.

In the illustrated form, the shaft 102 comprises an elongate body extending between the connection end 103 and the leading end 104. A delivery point 111 is located intermediate the connection end 103 and the leading end 104. The delivery point in the illustrated form comprises an angled tube extending outwardly from the shaft 102. The delivery point includes an injection connection located at the end of the angled tube. In other forms the delivery point may simply be an injection connection or may be a sealable aperture in the tube.

The shaft further comprises a fly wheel 114. The fly wheel 114 allows for rotation of the shaft. This allows the shaft 102 to be rotated with respect to the suction connector 103 and oriented for the preferred angle. In some forms, shaft 102 comprises a shaft stopper and rotator mechanism.

In some forms, the shaft 102 can rotate up to 360 degrees in either clockwise or anti clockwise directions giving flexibility to surgeons to deliver and remove components from the surgical site simultaneously.

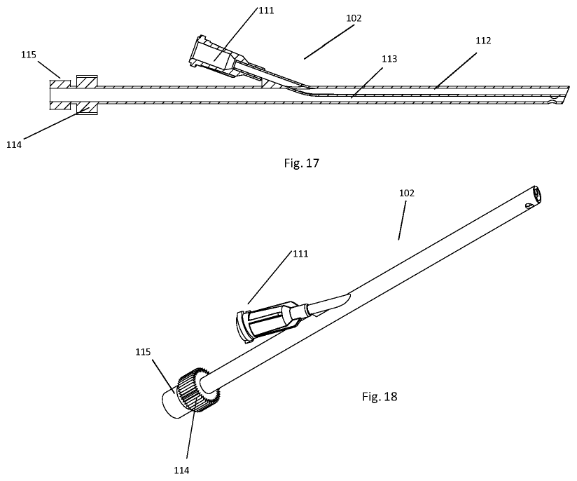

As shown best in the cross-section in FIG. 17, in some forms, the shaft 102 further comprises hollow closed section flow channels contained within the shaft 102. In the illustrated form a delivery flow channel 112 is configured for the delivery of components to the operational site. The delivery flow channel 112 is hollow and closed and has a circular or oval cross section but it will be clear that alternative configurations are available. The delivery flow channel 112 extends from the delivery point 111 to the leading end 104 and has a consistent cross section. In the illustrated form the delivery flow channel has a substantially circular cross section as shown in FIG. 16. In this form the delivery flow channel extends only approximately half the length of the suction flow channel 113. The delivery flow channel may extend any length with respect to the shaft.

The remainder of the interior of the shaft 102 comprises a suction flow channel 113 for the suction of the components from the surgical site. In the illustrated form the suction flow channel comprises an arched hollow closed section located around the delivery flow channel. However it will be clear that the delivery and suction flow channels may each be round tubes, may each be interior the shaft or may be engaged to one another and extend parallel to one another. Alternative configurations that allow for a delivery flow channel and a suction flow channel to extend from a leading end in a single device also fall within the scope of the disclosure.

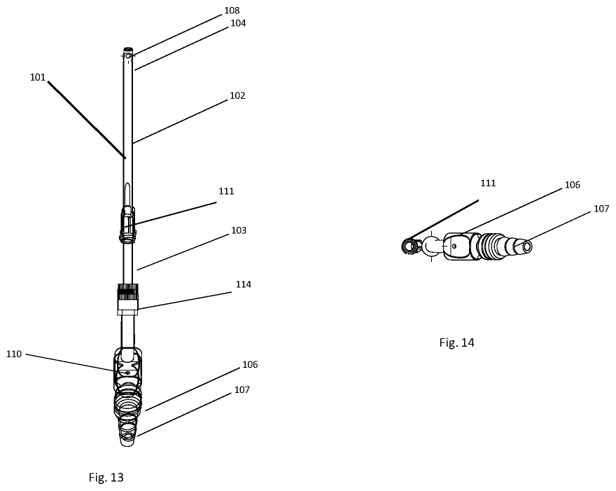

In the illustrated form, the shaft 102 comprises a small number of fenestrations 108 at the leading end as seen best in FIGS. 12 and 13. The fenestrations are of round shape, but they could be of any other shape and/or sizes. The fenestrations assist the suction and resist blockage at one point of the leading end. In the illustrated form, four spaced apart fenestrations 108 are located at the leading end 104.

In use, the device 101 is used to provide simultaneous delivery and suction. During a surgical procedure, a surgeon or other user positions the leading end 104 of the shaft 102 at a surgical site and connects the device with a source of suction. When a user's thumb is over the suction hole 110 suction occurs through the device and suction is provided from the suction flow channel 113.

The delivery point 106 is used to deliver a second component which then flows through the delivery flow channel 112 to the surgical site. The delivery point may be configured to allow injection of a second component or alternative delivery. In alternative embodiments a delivery device such as a syringe may be incorporated into the device.

During a procedure, suction and delivery can be independently controlled by the user allowing for simultaneous or alternate suction and delivery as needed. The user may also control the position and angle of delivery and suction by moving the shaft or by rotating the shaft.

The device has the benefit of providing a surgeon with a single-use dual cannulated articulated device that allows for both delivery of necessary active substance while removing blood and bodily fluid.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

What is claimed is:

1. A medical device for suction and delivery, the device comprising:
a shaft comprising a suction channel having a leading end for suctioning at least a first component from a surgical site, a delivery channel associated with the shaft, the delivery channel having an outlet for delivering a second component to the surgical site,
the suction channel leading end and the delivery channel outlet being independent from one another and located proximal to one another such that suction and delivery occur in relation to the same surgical site
the device configured such that in use during surgery the first component is removed from the site through the suction channel, and the second component is delivered to the site through the delivery channel, the device being configured such that in use removal of the first component and the delivery of the second component may occur simultaneously.

2. A device as defined in claim 1, wherein the first component comprises any of a fluid, a solid, or a semi solid mixture.

3. A device as defined in claim 1, wherein the second component comprises a haemostatic solution in the form of a fluid or solid powder.

4. A device as defined in claim 1, further comprising a flywheel for rotating the shaft with respect to a suction connector, the suction connector configured to be engaged with the suction channel to effect suction through the suction channel.

5. A device as defined in claim 1, further comprising a delivery connector at a delivery point to be engaged with the delivery channel to effect delivery through the delivery channel.

6. A device as defined in claim 1, wherein the suction channel and the delivery channel comprise hollow closed sections.

7. A device as defined in claim 3, wherein the hollow closed sections are located within the shaft.

8. A device as defined in claim 1, wherein the shaft further comprises fenestrations extending into the suction channel.

9. A device as defined in claim 1, wherein the device is configured such that suction of the first component and delivery of the second component are independently controlled.
